(19) United States
(12) Patent Application Publication
(10) Pub. No.: US 2002/0133061 A1
(43) Pub. Date: Sep. 19, 2002

(54) System and method for processing ventilator information

(76) Inventor: Amy M. Manetta, North Billerica, MA (US)

Correspondence Address: Siemens Corporation, Intellectual Property Department, 186 Wood Avenue South, Iselin, NJ, 08830, US

(21) Appl. No.: 09/805,970

(22) Filed: Mar. 14, 2001

Related U.S. Application Data
(60) Provisional application No. 60/248,087, filed on Nov. 13, 2000.

(51) Int. Cl. A61B 5/00

(52) U.S. Cl. 600/300

(57) ABSTRACT

An internet compatible system and method are described for displaying medical information derived from a plurality of sources. Ventilation unit parameters and/or settings associated with a patient are acquired on a substantially periodic basis and in response to a user command. The received ventilation unit parameters and or settings are prioritized for display in a desired order. An attribute is allocated to distinguish newly acquired ventilation unit parameters and/or settings that have changed from older ventilation unit parameters and settings.

### CROSS REFERENCE TO RELATED APPLICATION

This application claims the benefit of a provisional U.S. application, U.S. Serial No. 60/248,087 by A. M. Manetta, filed Nov. 13, 2000.

### FIELD OF THE INVENTION

This invention is related to the processing and displaying of medical information, and more particularly to processing and displaying of ventilator data in a network environment.

### BACKGROUND OF THE INVENTION

Ventilators are commonly used to ventilate a patient's lungs with breathing gas, so as to assist a patient when the patient's ability to breathe on his or her own is somehow impaired. In order to properly administer the ventilator, a caregiver must first set up various settings for the ventilator. Examples of commonly required settings to control a ventilator include: Peak Inspiratory Pressure (PIP) setting—limiting the peak pressure during inspiration of air; and Positive End Expiratory Pressure (PEEP) setting—limiting the peak pressure at the end of expiration of air. Many other ventilator settings may also be controlled, depending on the capability of the particular ventilator.

In addition, some ventilators are equipped with various sensors so that a patient caregiver may monitor the condition of the patient through the ventilator. Examples of commonly monitored parameters for a ventilator include Mean Airway Pressure (MAP)—the mean pressure measured within the airway during the breathing cycle; and Tidal Volume Inspired (TVi)—measured volume of gas inhaled by the patient during a normal breath. Many other ventilator parameters may also be monitored, depending on the sophistication of the ventilator.

### SUMMARY OF THE INVENTION

The present inventors recognize that as more knowledge is gained about the respiratory therapy and the number of settings and parameters that can be controlled and monitored increase with technological advances, there is a great need for a user-friendly and efficient way to process and display ventilator settings and parameters.

In addition, the present inventors recognize the desirability of a user being able to gather, process and display data remotely from a ventilator at any location and to use commonly available computing equipment, through for example, a local area network and/or a wide area network, such as the internet. Also, it is desirable for a device to be able to process and display not only the ventilator data from a particular ventilator, but also from other medical devices such as an anesthesia system or another ventilator on the same network.

Therefore, an internet compatible system and method are presented for displaying medical information derived from a plurality of sources. Ventilation unit parameters and/or settings associated with a patient are acquired on a substantially periodic basis and in response to a user command. The received ventilation unit parameters and or settings are prioritized for display in a desired order. An attribute is allocated to distinguish newly acquired ventilation unit parameters and/or settings that have changed from older ventilation unit parameters and settings.

In another aspect, when ventilator data are being acquired periodically and not in response to a user request, ventilator data will be stored only if one value of an acquired ventilator setting has changed. This is advantageous in preventing, for example, sometime frequent and inconsequential changes in a ventilator parameter to obscure more important changes in a ventilator setting.

### BRIEF DESCRIPTION OF THE DRAWINGS

In the drawing:

FIG. 1 is a block diagram of a communication network with various devices, according to the principles of the invention.

FIGS. 2A and 2B represent flow diagrams of a system according to the present invention.

FIG. 3 shows ventilator data being displayed according to the present invention.

FIG. 4 is an exemplary way to input ventilator parameters and settings using a web browser according to the present invention.

FIG. 5 shows an exemplary way of how a user may customize ventilator data displayed according to the principals of the invention.

FIG. 6 shows how customized data are being displayed.

### DETAILED DESCRIPTION

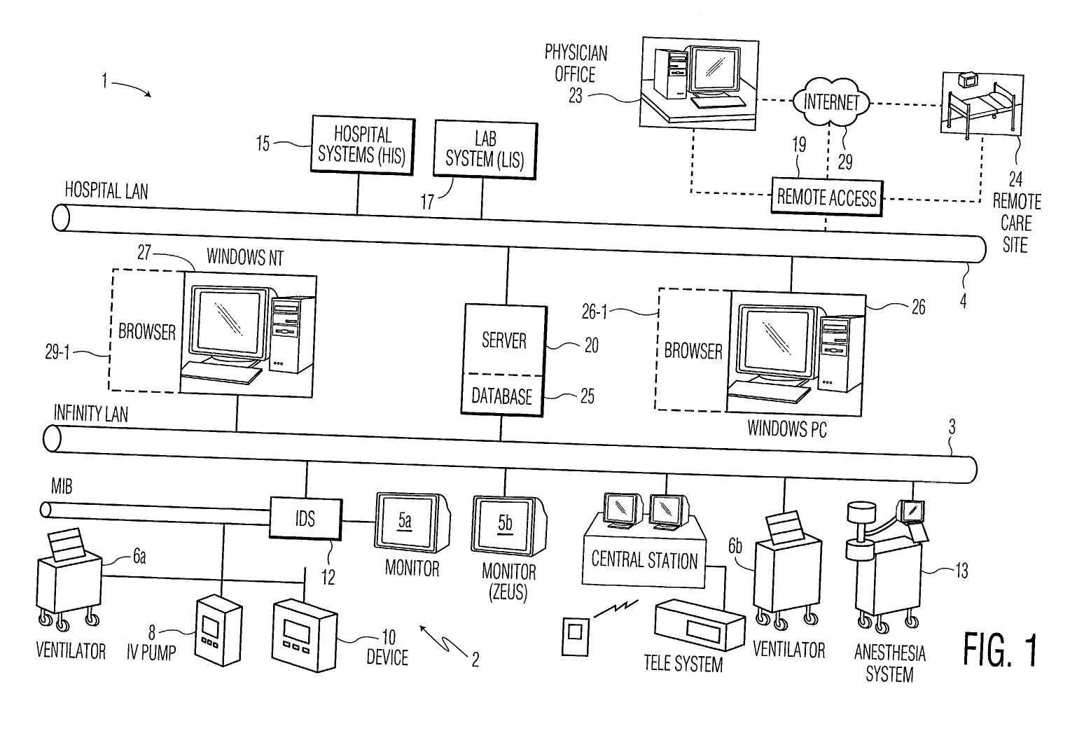

FIG. 1 is an exemplary block diagram of a communication network according to the principles of the present invention. As shown in FIG. 1, communication network 1 is represented by an IP (Internet Protocol) compatible network with a hierarchy of local area and wide area networks interconnected together. It is to be noted that although the present exemplary hospital or medical network is an IP compatible network, other types of network such as, but not limited to optical or wireless networks, using other computing protocols such as, but not limited to, for example, X.25, frame relay, IBM SNA etc., may also be used, as one skilled in the art can readily appreciate. In addition, although the exemplary network described is a hierarchical network, this is not required by the present invention. Any type of network architecture that provides communication connectivity among the devices on the network may be used.

As shown on FIG. 1, the first level of the exemplary hierarchical network 1 comprises a Medical Interface Bus (MIB) 2. A MIB is a well-known medical industry standard for locally connecting medical devices together. As shown in FIG. 1, MIB 2 is typically used to interconnect medical devices in a patient's room to administer care to a particular patient and to monitor the particular patient. Various medical devices may be connected via MIB 2; examples shown in FIG. 1 comprise a ventilator 6a, IV (Intravenous) Pump 8 or other medical equipment 10.

MIB 2 is typically connected to a second level LAN network 3 through an Interface Docking Station (IDS) device 12, for interfacing to Ethernet-compatible LAN network 3. The higher-level LAN 3 may be for example, an Infinity LAN, marketed by Siemens Medical System. This higher-level LAN 3 is typically, though not necessarily, used by a particular department within a hospital, such as an intensive care department or surgery department, etc., depending on the size of the organizations.

Although not shown in FIG. 1, more than one MIB may be connected to the second level LAN 3, so that more than one patient may be monitored or given care through LAN 3. In addition, medical devices may be connected directly to higher-level LAN 3. For example, as shown in FIG. 1, a ventilator 6b and an anesthesia system 13 are connected directly to LAN 3, without the need to go through a MIB.

Furthermore, LAN 3 may be interconnected to a Hospital LAN backbone 4 which also is Ethernet compatible. This backbone network 4 provides communication connectivity between various departments within a hospital or medical organization; for example, connecting hospital administrative systems 15 together with laboratory systems 17. In addition, the Hospital LAN 4 has a remote access gateway 19 which provides remote, secured access from, for example, a remote doctor's office 23 or a remote care site 24, to the various systems and devices on network 1, through for example, Internet 29. Alternatively, a remote site may also access the remote access gateway 19 directly through, for example, a dial-up telephone port, ADSL, or other types of private connection. Remote access gateway 19 may also be part of server 20, to be described below, instead of standing alone, as well know in the art.

According to the principles of the present invention, a central server 20 resides on LAN 3 for gathering and processing data from ventilators and other medical devices on network 1 for display and control. One skilled in the art can readily recognize that server 20 may reside at any level of the hierarchy of network 1, since all the different levels of LANs (e.g., 3, or 4), as well as remote sites in FIG. 1 are interconnected together. An example of server 20, is a Prometheus server, marketed by Siemens Medical System. The server may be hosted, for example, by a computer system that is capable of running Microsoft NT operating system.

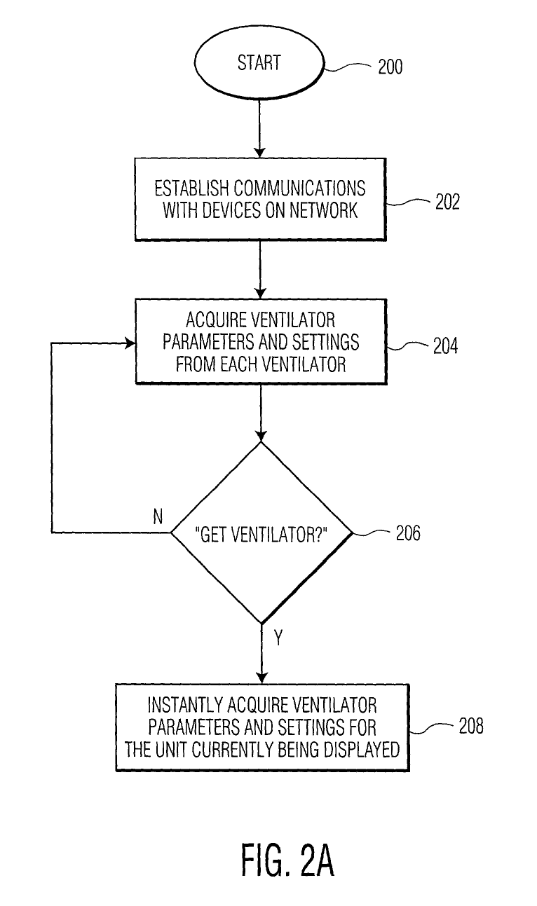

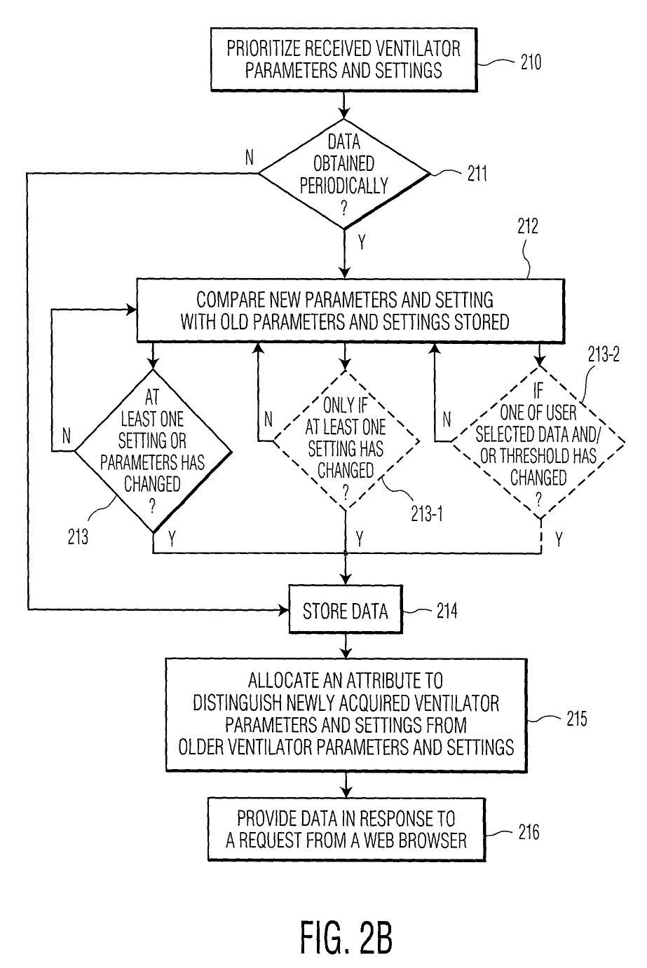

FIGS. 2A and 2B show in flow chart form, functions that may be performed by server 20 in accordance with the present invention. Server 20 first establishes communications with devices on the network as shown in step 202. This is done, for example, by using IP protocol and the known IP device address for each device on the network 1, in conjunction with a higher application-layer protocol, as well known in the art.

Once communications are established between server 20 and the other devices, server 20 starts to acquire parameters that are being monitored and settings selected for each ventilation unit (for example, 6a or 6b on network 1).

There are two different ways ventilator unit parameters and settings may be acquired by server 20 from each ventilator 6a or 6b. In step 204, ventilator data are periodically acquired from each ventilator 6a or 6b automatically. The periodically acquired data are then stored in a database 25 within the server 20. In addition, step 206 shows that a “get ventilator” request may be received by server 20 from, for example, a user computer 26 to be described in more detail later. In this case, server 20 will instantly acquire new ventilation unit parameters and settings for the unit currently being viewed by user computer 26, without waiting for the current update period to expire, as shown at step 208. This “get ventilator” feature is particularly useful when critical, real time data are needed to make quick decisions, without having to wait for the next periodic update.

In one aspect of the present invention, a user may use a Microsoft Windows compatible PC 26 or Windows NT compatible PC 27 as shown in FIG. 1, or any other computers capable of running a menu generating program such as a web browser program (e.g., Microsoft Internet Explorer or Netscape Navigator, etc.) to monitor ventilator parameters and settings. That is, a user may use a web browser on any computer, as long as a communication connection can be made to server 20, to make request and view information to and from a ventilator on network 1 through server 20. This is advantageous, since a doctor may for example, gain access to a particular ventilator from, for example, a remote physician's office 23, without having to access a dedicated terminal. Of course, a user can simply use a keyboard and/or a mouse or any other user interface devices to enter a user selection or request on a user computer, as is known in the art.

Server 20 is therefore capable of formatting ventilator data to be compatible with, for example, HTML (HyperText Mark-up Language) programming language for displaying data on a web browser. The server is also responsive to, for example, HTTP (HyperText Transfer Protocol) commands originated from a user's web browser for making a request.

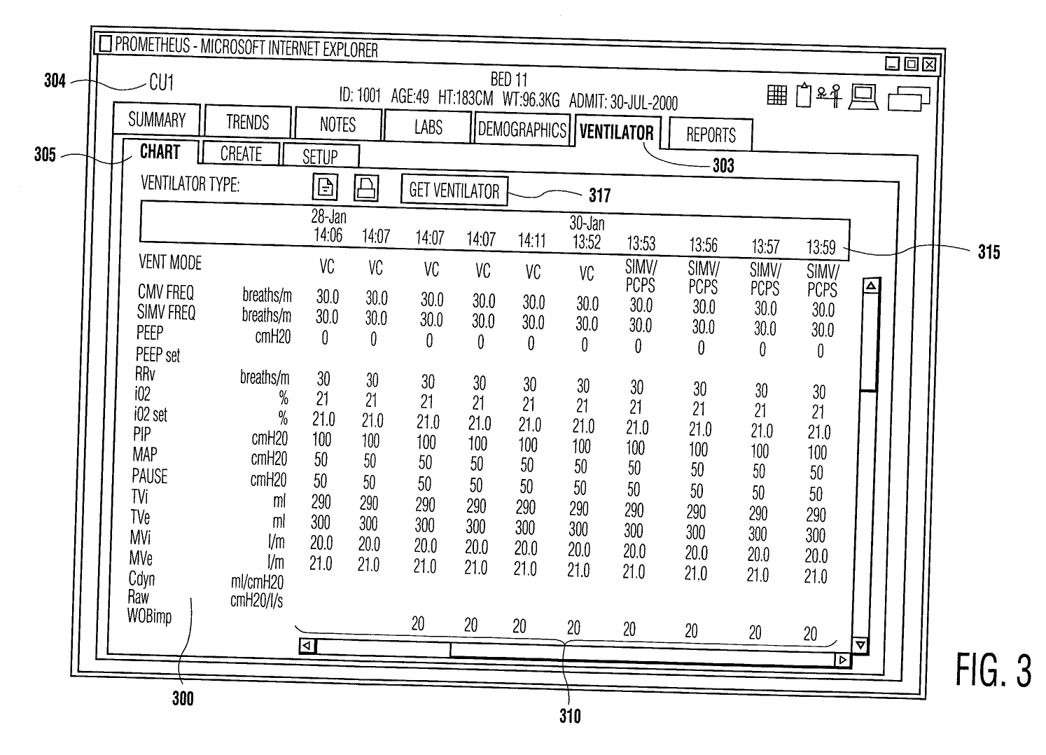

FIG. 3 shows an example of how ventilator settings and parameters may be displayed on a web browser of a user computer 26, according to the present invention. A user may request access to a particular ventilator by, for example, specifying the name of a particular patient or bed on the network (e.g., CU1 304, Johnson or Bed 11) and by selecting on ventilator tab 303. An exemplary chart display 300 is shown in FIG. 3 when the user selects chart icon 305. Exemplary chart 300 displays, on the left most column, names of the ventilator unit parameters and settings being displayed. The values of these parameters and settings are shown in the rest of the columns 310 in time sequence order. The time when each value was sampled is specified in the upper row 315. As described before, a “get ventilator” function may be requested to obtain ventilator data. This function may be requested by user selecting “get ventilator” icon 317 in FIG. 3. In one aspect according to the present invention, “get ventilator” icon 317 will only be active and capable of being selected on user computer 26 when the specified ventilator is recognized on hospital network 1 by server 20.

The displayed ventilator data are additionally processed by server 20 as described in FIG. 2B. As shown in step 210 of FIG. 2B, once ventilator unit data are obtained from a particular ventilator unit such as ventilator 6a or 6b shown in FIG. 1, either instantly or periodically as described before, server 20 will prioritize the received ventilation unit parameters and settings for the particular ventilator. The server prioritizes the ventilator data in response to user request and customization of data on a web browser on, for example, computer 26 to be described in more detail below.

In step 212, if data are obtained periodically, server 20 will compare newly acquired parameters and settings with existing or old parameters and settings stored in database 25. New data will be stored in database 25 for display only if at least one ventilator setting or parameter has changed, as shown in steps 213 and 214. This would allow more efficient use of database and bandwidth. However, if data are obtained in response to “get ventilator” command, Server 20 will store the data, without doing any comparison to see whether data have changed or not, as shown in steps 211 and 214.

In one aspect according to the principles of the invention, the present inventors recognize that ventilator parameters tend to change frequently (for example, TVi may changed for each inhalation by a patient), but on the other hand, ventilator settings tend to change infrequently. Therefore, present inventors recognize that it may be more informative and instructive for a caregiver if data are displayed periodically (i.e., with changes highlighted) only if at least one of the ventilator settings, not parameters have changed. Therefore, in one alternative embodiment of the present invention, as shown in step 213-1, ventilator data will only be stored for displayed, if at least one ventilator setting has changed, regardless of whether any of the ventilator parameters has changed.

In another embodiment, shown in step 213-2, in order to provide further flexibility, the user is allowed to set the types of comparison of data server 20 will use to determine what data are to be stored into database 25. Additionally, the user may not only select the types of ventilator data to be compared (for example, parameters and/or settings), he or she may also be allowed to specify a threshold of change. That is, a user is allowed to select or enter, for example, a threshold percentage number (e.g., 10%) so that only when selected newly acquired data have exceeded this threshold will the newly acquired data be stored for display. This provides the user with even more efficient and customizable display of ventilator data.

In step 215, server 20 will then allocate an attribute to distinguish newly acquired ventilation unit parameters and settings that have changed from older ventilation unit and parameters and settings. One exemplary attribute may be display color. That is, when the ventilator chart shown in FIG. 3 is requested to be displayed via computer 26, ventilator data will be color coded on the web browser so that the user is able to distinguish what new data have changed. For example, as old data that are displayed on the screen in one color (e.g., black) and carried forward to the left in each column as time advances, any newly acquired data that have changed will be displayed in another color (e.g., blue), in the column representing the current time.

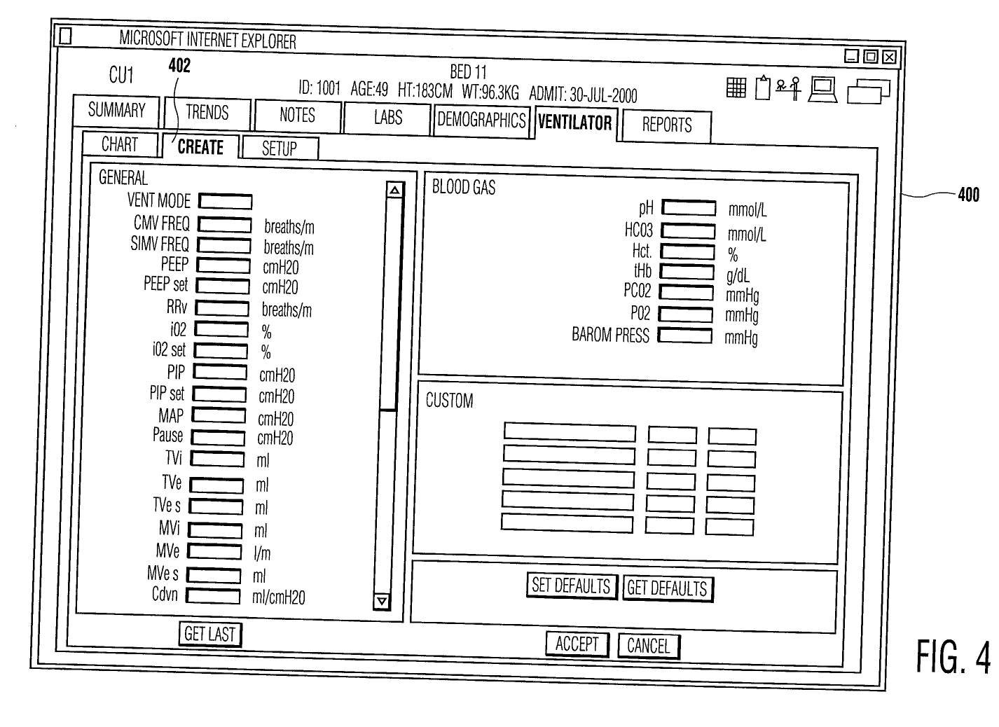

In another aspect of the present invention, customization of data may be provided through a web browser on a user computer, in response to a user request. In one exemplary embodiment, a user customization screen 400 such as that shown in FIG. 4 may be invoked by a user selecting an icon “create” 402 on his or her browser. As shown in screen 400, various ventilator parameters and settings for a selected ventilator will then be displayed with value of each parameter or setting being left blank. This will allow the user to enter a particular value so that, for example, selected settings for a selected patient may be entered remotely using a web browser.

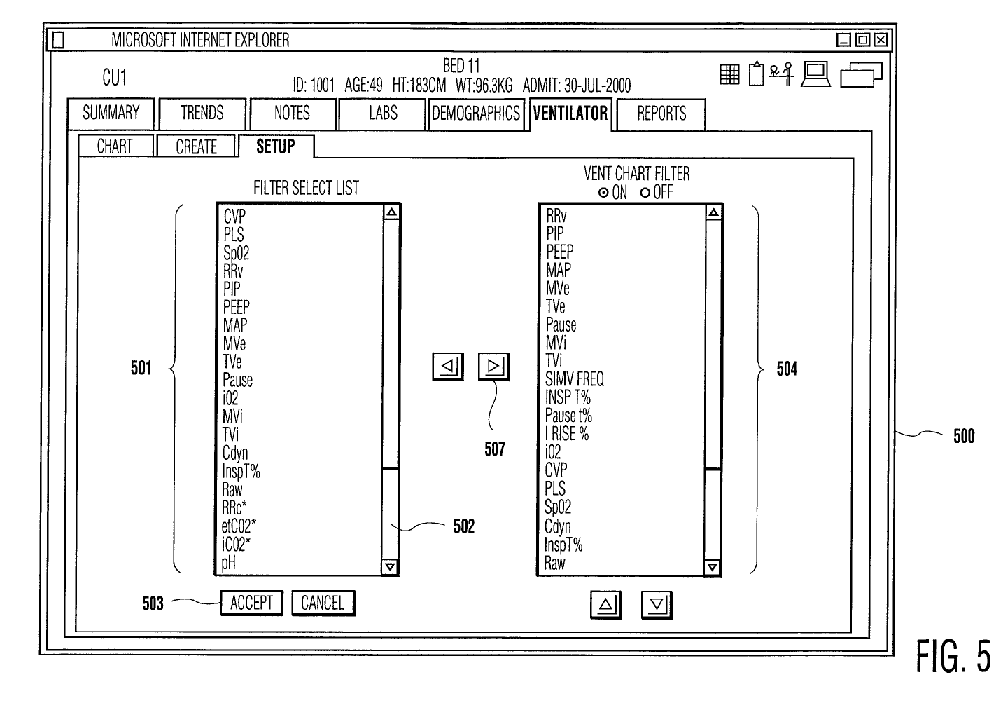

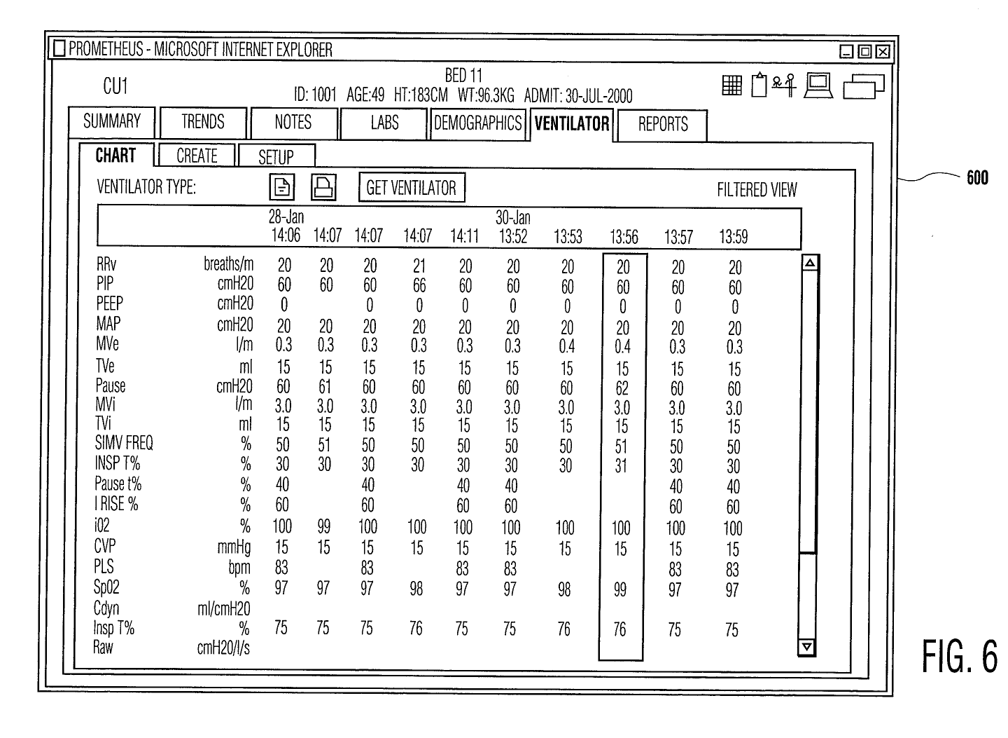

Additionally, a user may “filter” what parameters and settings are displayed on his or her browser screen when chart option 304 (FIG. 3) is selected as described before. FIG. 5 shows an exemplary browser screen 500 for accomplishing the “filter view” function. A filter select list window 501 showing a list of possible parameters and settings for a ventilator is displayed. The user may first, for example, highlight any one of the names in list 501 and then click on the right arrow key 507. The selected parameters or settings will then be added to the vent chart filter screen 504 for display. Once all the selections have been made and the “filtered view” screen is selected, only the selected or filtered parameters and settings will be displayed on the web browser screen as shown on for example, screen 600 of FIG. 6. This feature allows the user to easily and efficiently customize his or her data viewing according to his or her needs.

It is to be understood that the embodiments and variations shown and described herein are for illustrations only and that various modifications may be implemented by those skilled in the art without departing from the scope of the invention.

What is claimed is:

1. An internet compatible system for displaying medical information derived from a plurality of sources, comprising:
a communication network for acquiring ventilator parameters associated with a patient on a substantially periodic basis and in response to a user command; and
a device for prioritizing received ventilator parameters for display in a desired order and for allocating an attribute to distinguish changed ventilator parameters.

2. The system of claim 1 wherein the attribute is a different color.

3. The system of claim 2 wherein the communication network further acquires ventilator settings, as well the parameters; and the device further prioritizes received ventilator settings, as well as the received parameters.

4. The system of claim 3 further comprising a menu generator for generating a window for displaying said ordered ventilator parameters and settings in a first window.

5. The system of claim 4 wherein the menu generator is an internet browser.

6. The system of claim 4 wherein the ventilator parameters and settings are displayed so that the changed ventilator parameters and changed ventilator settings are displayed in the different color.

7. The system of claim 3 wherein the device, in response to the user command, acquires a new set of ventilator parameters and settings.

8. The system of claim 3 wherein the device prioritizes the received ventilation unit parameters and settings for display in a desired order in response to a second user command.

9. The system of claim 8 wherein the second user command comprising selection of a filtered list.

10. The system of claim 8 wherein the second user command comprising creation of a set of values for selected parameters and settings.

11. The system of claim 4 wherein said menu generator comprises a user selection for selecting any one of the plurality of sources.

12. An internet compatible method for displaying medical information derived from a plurality of sources, comprising the steps of:
acquiring ventilator parameters associated with a patient on a substantially periodic basis and in response to a user command; and
prioritizing received ventilator parameters for display in a desired order and for allocating an attribute to distinguish changed parameters.

13. The method of claim 12, wherein the attribute is a different color.

14. The method of claim 13 wherein the acquiring step further comprising acquiring ventilator settings, as well the parameters; and the prioritizing step further comprising prioritizing received ventilator settings, as well as the received parameters.

15. The method of claim 14 further comprising the step of generating a window for displaying said ordered ventilator parameters and settings.

16. The method of claim 15 wherein the generating step is done by an internet browser.

17. The method of claim 15 wherein the generating step displays the ventilator parameters and settings so that the changed ventilator parameters and changed ventilator settings are displayed in the different color.

18. The method of claim 14 further comprising the step of acquiring another set of new ventilation unit parameters and settings, in response to the user command.

19. The method of claim 14 wherein the step of prioritizing the received ventilation unit parameters and settings for display in a desired order is in response to a second user command.

20. The method of claim 19 wherein the second user command comprising selection of a filtered list.

21. The method of claim 19 wherein the second user command comprising creation of values for selected parameter and settings.

22. The method of claim 12 further comprising the step of selecting any one of a plurality of sources.

23. A method for acquiring and storing ventilator data comprising ventilator parameters and ventilator settings from a medical device over a communication network, comprising the steps of:
establishing communication with the medical device over the communication network;
acquiring selected ventilator data from the medical device over the communication network;
determining if a value of at least one of: 1) ventilator settings and 2) ventilator parameters of acquired ventilator data has changed; and
if the value has changed, storing the acquired ventilator data.

24. The method of claim 23, wherein if the selected ventilator data are acquired in response to a user request, automatically storing the acquired ventilator data, without the determining step.

25. The method of claim 23 further comprising the step of allocating an attribute to distinguish any changed ventilator data from previously acquired ventilator data.

26. The method of claim 23 further comprising the step of determining if the value has changed more than a predetermined threshold.

27. A method for acquiring and storing ventilator data comprising ventilator parameters and ventilator settings from a medical device over a communication network, comprising the steps of:
establishing communication with the medical device over the communication network;
acquiring selected ventilator data periodically from the medical device over the communication network;
determining whether a value of ventilator settings of acquired ventilator data has changed; and
if the value has changed, storing the acquired ventilator data.
